(19) United States
(12) Patent Application Publication
(10) Pub. No.: US 2023/0348994 A1
(43) Pub. Date: Nov. 2, 2023

(54) Identification of Immune-related lncRNA Signatures with Prognostic Connotation in Multi-Cancer Setting

(71) Applicants: Qatar Foundation for Education, Science and Community Development, Doha (QA); Sidra Medicine, Doha (QA)

(72) Inventors: Julie Valerie Caroline M Decock, Doha (QA); Wouter Hendrickx, Doha (QA); Raghvendra Mall, Doha (QA)

(21) Appl. No.: 18/141,167

(22) Filed: Apr. 28, 2023

Related U.S. Application Data
(60) Provisional application No. 63/336,576, filed on Apr. 29, 2022.

(51) Int. Cl. C12Q 1/6886

(52) U.S. Cl. CPC C12Q 1/6886; C12Q 2600/178; C12Q 2600/158; C12Q 2600/112

(57) ABSTRACT

The present disclosure provides a method comprised of one or a combination of ir-lncRNA signatures to predict the clinical outcome of cancer patients, improve prognostic stratification of patients and guide cancer treatment decisions.

### CROSS REFERENCE TO RELATED APPLICATION

This application claims the benefit of, and priority to, U.S. Provisional Patent Application Ser. No. 63/336,576, filed on Apr. 29, 2022, the entire disclosure of which is hereby incorporated by reference in its entirety.

### BACKGROUND

The clinical outcome and treatment response of cancer patients is greatly influenced by the host immune response and its interactions with the tumor. Immunotherapy (immune-based therapy) has transformed standard clinical cancer care and shown very promising results, however, thus far its success is limited to a select group of patients and tumor types.

Therefore, studies have focused on identifying gene signatures that can predict the immune contexture of a tumor, and thus the likelihood of a greater treatment response. Although several prognostic immune gene signatures have been identified, their applicability is limited to few specific cancer types.

A 2007 study found that only one-fifth of transcription across the human genome is associated with protein-coding genes, indicating the presence of at least four times more non-coding than coding RNA sequences. RNAs longer than 200 nucleotides that are not translated into functional proteins are known as long non-coding RNAs (lncRNAs).

The human genome contains more than 18,000 lncRNAs and over 51,000 lncRNA transcripts. The resulting lncRNAs are often capped by 7-methyl guanosine (m⁷G) at their 5′ ends, polyadenylated at their 3′ ends, and spliced similarly to mRNAs.

LncRNAs are involved in numerous biological processes regulating gene expression and post-transcriptional modification and have been implicated in many diseases including cancer.

Several immune-related lncRNA signatures have been identified with prognostic connotations for specific cancer types, including gastric cancer, breast cancer, head and neck cancer, cutaneous melanoma, lung cancer, colorectal cancer, bladder cancer and hepatocellular carcinoma. However, the prognostic value of these lncRNA signatures are likely limited to the tumor type in which they were identified and may not be applicable to patient cohorts of diverse ancestral origin.

Therefore, improved methods are needed to provide increased prognostic capability.

### SUMMARY

The present disclosure generally relates to the identification and use of immune related lncRNA signatures having prognostic connotations in numerous cancer types, and in patients of diverse ancestral origin.

Disclosed herein are methods to identify immune related lncRNA signatures in tumor tissues.

Disclosed herein are methods of use of immune related lncRNA signatures to predict clinical outcomes of cancer patients.

Further methods comprise development of a treatment protocol based on the predicted clinical outcome.

The present disclosure also relates to the identification of correlations between immune related lncRNA signatures and immune checkpoint molecules.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 depicts the workflow that is used to identify immune-related lncRNA signatures with prognostic connotation in various cancers and their proxy protein-coding gene networks and signaling pathways.

FIG. 2 depicts breast cancer immune-related lncRNAs and associated biological pathways and functions. FIG. 2A depicts the volcano plot of differentially expressed lncRNAs in immune favorable vs unfavorable breast tumors from the TOGA breast cancer cohort. FIG. 2B depicts a representation of the number of differentially expressed lncRNAs by ICR in TOGA-BRCA and their proximal protein-coding genes as per the RWR propagation algorithm. FIG. 2C depicts pathway enrichment analysis of proxy protein-coding genes using consensus PathDB. For each pathway, the number of differentially expressed genes is indicated and the proportion of up- and downregulated genes in ICR high vs ICR low is visualized in red and green respectively. FIG. 2D depicts IPA analysis of diseases and functions associated with top-ranked proxy protein-coding genes.

FIG. 3 depicts a comparison of the composition and prognostic value of three immune-related lncRNA signatures with the ICR signature in TOGA breast cancer patients. FIG. 3A depicts the heatmap of Spearman's rank correlation coefficients between the expression of ir-lncRNAs and 30 immune checkpoints, color-coded from −1 (dark blue) to +1 (dark red). FIG. 3B depicts the forest plots showing HRs (overall survival) of the continuous enrichment scores of different immune signatures with p-values in the TOGA breast cancer cohort. FIG. 3C depicts the composition and intersection of ir-lncRNAs of all three lncRNA signatures in TOGA-BRCA.

FIG. 4 depicts the prognostic significance of the 20-ICRlncRNA and 20-ICPlnRNA signature in solid cancers. FIG. 4A shows the top 20 differentially expressed ir-lncRNAs by ICR, that can predict the clinical outcome of cancer patients. FIG. 4B shows the top 20 differentially expressed ir-lncRNAs associated with immune checkpoints, that can predict the clinical outcome of cancer patients.

FIG. 5 depicts the prognostic significance of the 3 ir-lncRNAs signature in solid cancers. FIG. 5A depicts forest plots showing HRs (overall survival) of the enrichment scores of the 3 ir-lncRNA signature, p-values, and the number of patients for each TOGA cancer cohort and RAQA breast cancer cohort. FIG. 5B depicts overall survival Kaplan-Meier curves of selected cancers, dichotomized by the enrichment score of the 3 ir-lncRNAs signature.

FIG. 6 depicts Receiver Operating Characteristic (ROC) analysis of 3 ir-lncRNA and ICR signatures in selected solid cancers.

FIG. 7 depicts the mapping of differentially expressed ir-lncRNAs to protein coding genes. FIG. 7A depicts a diagram representation of the random walk with restart global propagation network algorithm. FIG. 7B depicts the walkscore distribution of protein-coding genes in TOGA-BRCA, with cutoff set at walkscore ≥0.01 to generate a ranked list of protein-coding genes in proximity of differentially expressed ir-lncRNAs.

FIG. 8 depicts the prognostic value of the ICR classifier across solid cancers.

FIG. 9 depicts survival curves of the 3 ir-lncRNA signature.

FIG. 10 depicts the ROC analysis of the 3 ir-lncRNA signature across multiple solid cancers.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used in this disclosure and the appended claims, the singular forms “a,” “an” and “the” include plural referents unless the context clearly dictates otherwise. Thus, for example, reference to “a component” or “the component” includes two or more components.

“Administration,” or “to administer” means the step of giving (i.e. administering) a pharmaceutical composition or active ingredient to a subject. The pharmaceutical compositions disclosed herein can be administered via a number of appropriate routes.

The words “comprise,” “comprises” and “comprising” are to be interpreted inclusively rather than exclusively. Likewise, the terms “include,” “including,” “containing” and “having” should all be construed to be inclusive, unless such a construction is clearly prohibited from the context. Further in this regard, these terms specify the presence of the stated features but not preclude the presence of additional or further features.

Nevertheless, the compositions and methods disclosed herein may lack any element that is not specifically disclosed herein. Thus, a disclosure of an embodiment using the term “comprising” is:
a. (i) a disclosure of embodiments having the identified components or steps and also additional components or steps;
b. (ii) a disclosure of embodiments “consisting essentially of” the identified components or steps; and
c. (iii) a disclosure of embodiments “consisting of” the identified components or steps. Any embodiment disclosed herein can be combined with any other embodiment disclosed herein.

The term “and/or” used in the context of “X and/or Y” should be interpreted as “X,” or “Y,” or “X and Y.” Similarly, “at least one of X or Y” should be interpreted as “X,” or “Y,” or “X and Y.”

Where used herein, the terms “example” and “such as,” particularly when followed by a listing of terms, are merely exemplary and illustrative and should not be deemed to be exclusive or comprehensive.

“Cancer” is a disease characterized by uncontrolled growth of cells. The embodiments disclosed herein may target any type of cancer that expresses LDHC or a similar molecule.

“LncRNAs” refers to long non-coding RNAs.

“FOR” or the polymerase chain reaction, is a chemical reaction used to amplify DNA sequences.

“Patient” means a human or non-human subject receiving medical or veterinary care.

“Pharmaceutically acceptable” or “therapeutically acceptable” refers to a substance which does not interfere with the effectiveness or the biological activity of the active ingredients and which is not toxic to a patient.

“Pharmaceutically acceptable carrier” is art-recognized, and includes, for example, pharmaceutically acceptable materials, compositions or vehicles, such as a liquid or solid filler, diluent, excipient, solvent, or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic.

Exemplary materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, sunflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

“Pharmaceutical composition” means a formulation comprising an active ingredient. The word “formulation” means that there is at least one additional ingredient (such as, for example and not limited to, an albumin [such as a human serum albumin or a recombinant human albumin] and/or sodium chloride) in the pharmaceutical composition in addition to a botulinum neurotoxin active ingredient. A pharmaceutical composition is therefore a formulation which is suitable for diagnostic, therapeutic or cosmetic administration to a subject, such as a human patient. The pharmaceutical composition can be in a lyophilized or vacuum dried condition, a solution formed after reconstitution of the lyophilized or vacuum dried pharmaceutical composition with saline or water, for example, or as a solution that does not require reconstitution. As stated, a pharmaceutical composition can be liquid, semi-solid, or solid. A pharmaceutical composition can be animal-protein free.

“Therapeutic formulation” means a formulation that can be used to treat and thereby alleviate a disorder or a disease and/or symptom associated thereof.

“Therapeutically effective amount” means the level, amount or concentration of an agent needed to treat a symptom, disease, disorder, or condition without causing significant negative or adverse side effects.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

The terms “tumor” or “tumour” or “cancer” are used interchangeably.

### EMBODIMENTS

The present disclosure generally relates to the identification and use of immune-related lncRNA signatures that have prognostic connotations in multiple cancer types, and in patients of diverse ancestral origin.

Thus, disclosed embodiments comprise methods of predicting clinical treatment outcomes, for example treatment outcomes in cancer patients.

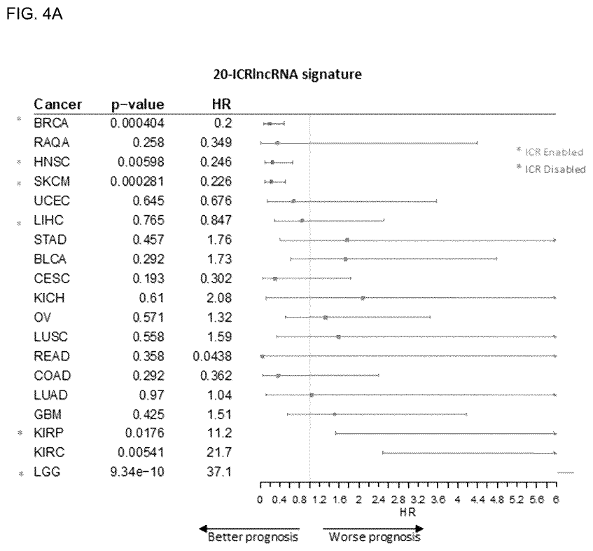

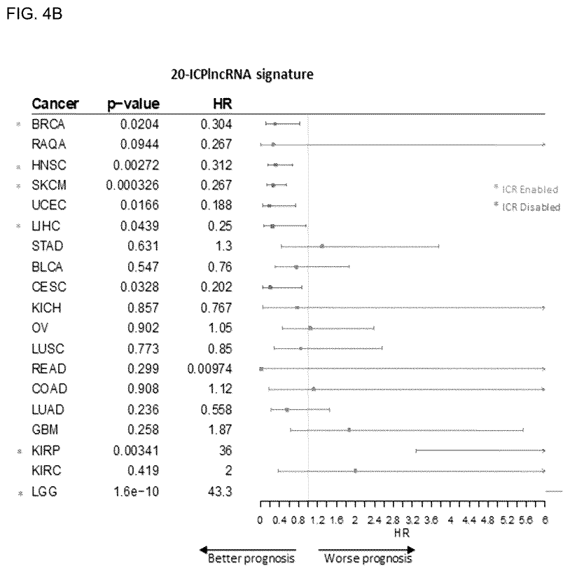

For example, in some embodiments of the present disclosure, the immune-related lncRNA signature is a 20-ICRlncRNA signature, comprising the top 20 differentially expressed ir-lncRNAs by ICR, that can predict the clinical outcome of cancer patients (FIG. 4A).

In other embodiments of the present disclosure, the immune-related lncRNA signature is a 20-ICPlncRNA signature, comprising of the top 20 differentially expressed ir-lncRNAs associated with immune checkpoints, that can predict the clinical outcome of cancer patients (FIG. 4B).

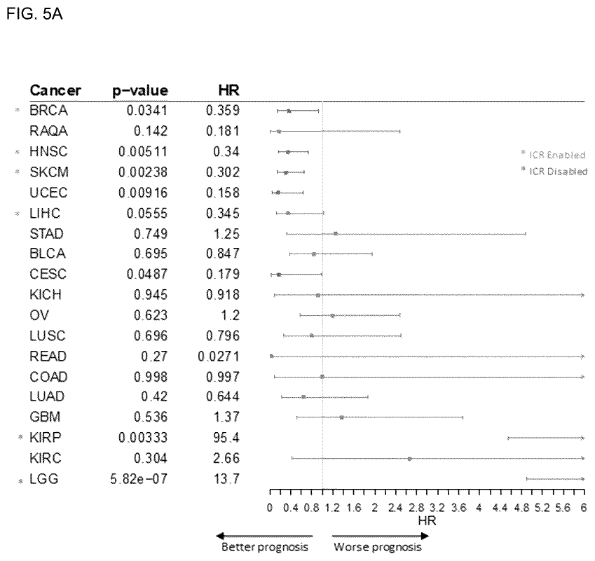

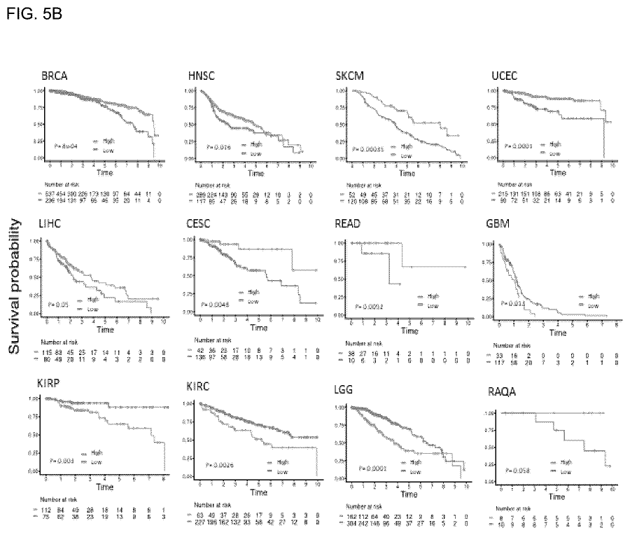

In some embodiments of the present disclosure, the immune-related lncRNA signature is a 3 ir-lncRNA signature, comprising of the overlap of both 20-lncRNA signatures, that can predict the clinical outcome of cancer patients including breast cancer, head and neck cancers, melanoma, uterine cancer, liver cancer, kidney cancer and low-grade glioma brain cancer (FIG. 5).

The present disclosure also relates to the identification of a specific combination of 3 immune-related lncRNAs (not the individual naturally occurring lncRNAs) that has translational potential as a prognostic and predictive tool to improve clinical decision making in 7 cancer types.

In some embodiments of the present disclosure, the 3-lncRNA signature performs equally well as (and in UCEC outperforms) the 20-gene ICR signature to predict the clinical outcome of cancer patients with the advantage that the lncRNA molecular signature is significantly less complex, featuring only 3 molecules.

The present disclosure also relates to the identification of ir-lncRNAs that correlate with immune checkpoint molecules which may indicate that these could be predictive of immune checkpoint expression and possibly immune checkpoint therapy response, a type of immunotherapy that block immune checkpoint proteins from binding with partner proteins.

In other embodiments of the present disclosure, the methods of mapping immune-related lncRNAs to a coding-non-coding network are described that can provide insight into the putative molecular mechanisms underlying ir-lncRNA prognostic and predictive value.

Disclosed embodiments comprise a prognostic and predictive molecular test for cancer, in analogy with for example the OncoType DX or MammaPrint micro-array assays for breast cancer.

In some embodiments of the present disclosure, the use of ir-lncRNA signatures can comprise a PCR-based assay to detect specific lncRNA expression.

In other embodiments of the present disclosure, the use of ir-lncRNA signatures can comprise a stand-alone test, as well as “array” tests using, for example a “Q-chip” device to detect ir-lncRNA expression.

In other embodiments of the present disclosure, the use of ir-lncRNA signatures can comprise a combination of any of the above-mentioned embodiments to detect ir-lncRNA expression. In addition, detection of ir-lncRNA expression can also be accomplished by other methods known to one of ordinary skill in the art.

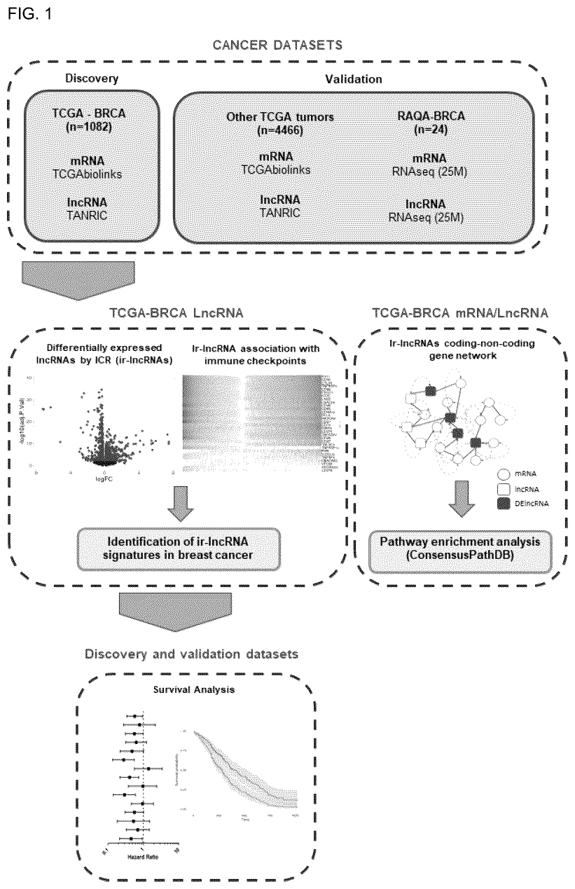

Turning to the Figures, FIG. 1 depicts the workflow that is used to identify immune-related lncRNA signatures with prognostic connotation in various cancers and their proxy protein-coding gene networks and signaling pathways.

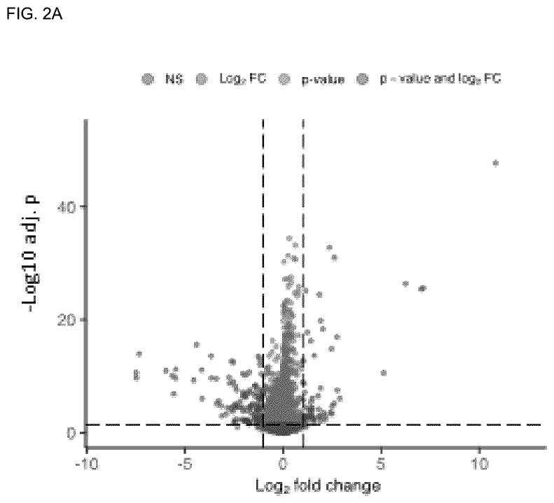

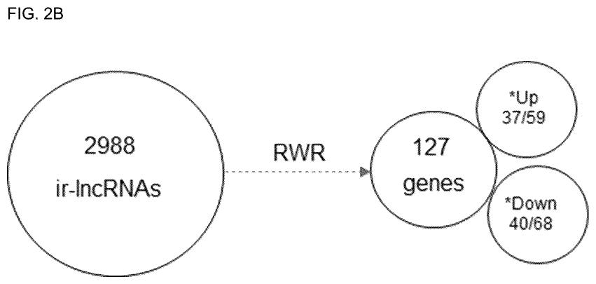

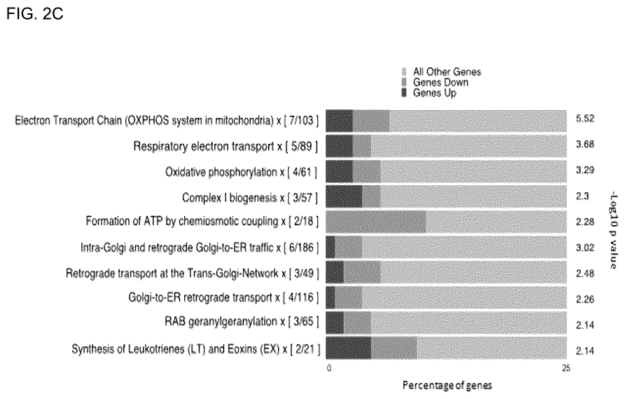

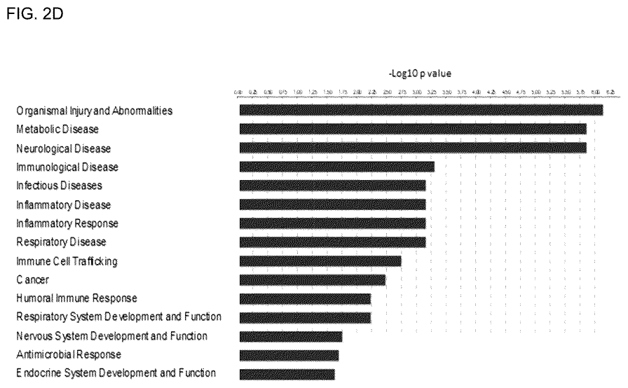

FIG. 2 depicts breast cancer immune-related lncRNAs and associated biological pathways and functions. FIG. 2A depicts the volcano plot of differentially expressed lncRNAs in immune favorable vs unfavorable breast tumors from the TOGA breast cancer cohort. The established immunologic constant of rejection (ICR) 20-gene signature was used to classify tumors as immune favorable (ICR high) vs unfavorable (ICR low). Red=log 2 fold change>1 and adj p-value<0.05, blue=adj p-value<0.05, and green=adj p-value>0.05.

FIG. 2B depicts a representation of the number of differentially expressed lncRNAs by ICR in TCGA-BRCA and their proximal protein-coding genes as per the RWR propagation algorithm. Protein coding genes with confirmed up- or downregulation as per differential expression analysis of RNAseq data are indicated with asterisks. The mRNA expression of 59 up- and 68 downregulated genes was available in the RNAseq data, and the differential expression of 37 and 40 genes was confirmed.

FIG. 2C depicts pathway enrichment analysis of proxy protein-coding genes using consensus PathDB. For each pathway, the number of differentially expressed genes is indicated and the proportion of up- and downregulated genes in ICR high vs ICR low is visualized in red and green respectively.

FIG. 2D depicts IPA analysis of diseases and functions associated with top-ranked proxy protein-coding genes.

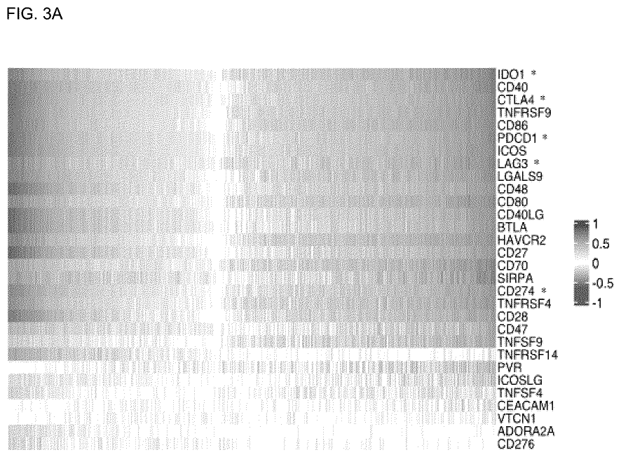

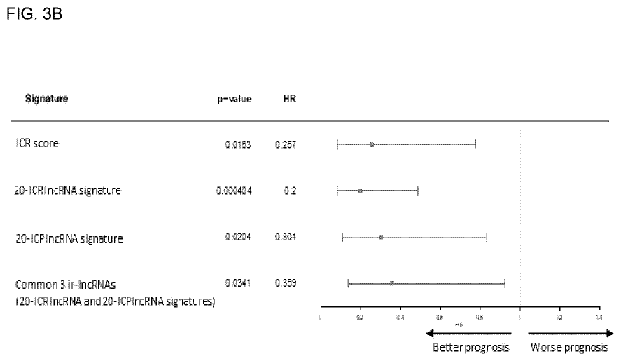

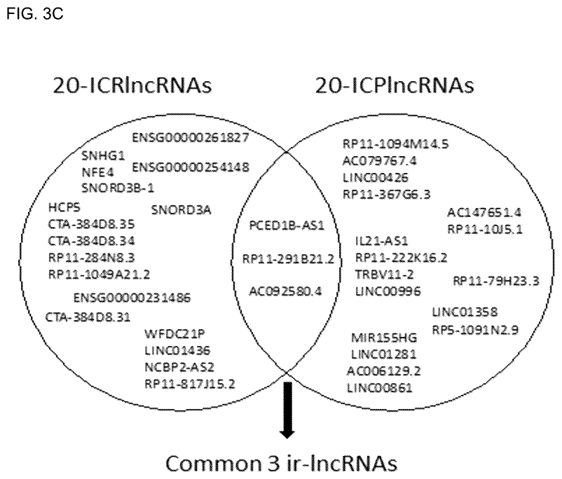

FIG. 3 depicts a comparison of the composition and prognostic value of three immune-related lncRNA signatures with the ICR signature in TOGA breast cancer patients. The first signature is comprised of the top 20 differentially expressed ir-lncRNAs by ICR (20-ICRlncRNA), the second of the top 20 differentially expressed ir-lncRNAs associated with immune checkpoints (20-ICPlncRNA), and the third signature comprises the overlap of both 20-lncRNA signatures.

FIG. 3A depicts the heatmap of Spearman's rank correlation coefficients between the expression of ir-lncRNAs and 30 immune checkpoints, color-coded from −1 (dark blue) to +1 (dark red). Columns are ordered by the sum of the correlation scores and rows are ordered by the absolute sums of the correlation scores. Immune checkpoints that are included in the ICR signature are indicated with a red asterisk.

FIG. 3B depicts the forest plots showing HRs (overall survival) of the continuous enrichment scores of different immune signatures with p-values in the TOGA breast cancer cohort. Significant negative HRs are visualized in red.

FIG. 3C depicts the composition and intersection of ir-lncRNAs of all three lncRNA signatures in TOGA-BRCA.

FIG. 4 depicts the prognostic significance of the 20-ICRlncRNA and 20-ICPlnRNA signature in solid cancers. Forest plots showing HRs (overall survival) of the enrichment scores of the (A) 20-ICRlncRNA and (B) 20-ICPlncRNA signature, p-values, and number of patients for each TOGA cancer cohort and the local RAQA breast cancer cohort. Significant positive HRs are visualized in blue and significant negative HRs are visualized in red. ICR enabled (HR<1, p-value<0.05) cancer types are indicated with orange asterisks and ICR disabled (HR>1, p-value<0.05) cancers are indicated with purple asterisks. BRCA, breast invasive carcinoma; RAQA, retrospective Arab Qatar cohort; HNSC, head and neck squamous cell carcinoma; SKCM, skin cutaneous melanoma; UCEC, uterine corpus endometrial carcinoma; LIHC, liver hepatocellular carcinoma; STAD, stomach adenocarcinoma; BLCA, bladder urothelial carcinoma; CESC, cervical squamous cell carcinoma and endocervical adenocarcinoma; KICH, kidney chromophobe; OV, ovarian serous cystadenocarcinoma; LUSC, lung squamous cell carcinoma; READ, rectum adenocarcinoma; COAD, colon adenocarcinoma; LUAD, lung adenocarcinoma; GBM, glioblastoma multiforme; KIRP, kidney renal papillary cell carcinoma; KIRC, kidney renal clear cell carcinoma; LGG, brain lower grade glioma.

FIG. 5 depicts the prognostic significance of the 3 ir-lncRNAs signature in solid cancers.

FIG. 5A depicts forest plots showing HRs (overall survival) of the enrichment scores of the 3 ir-lncRNA signature, p-values, and the number of patients for each TOGA cancer cohort and RAQA breast cancer cohort. Significant positive HRs are visualized in blue and significant negative HRs are visualized in red. ICR enabled (HR<1, p-value<0.05) cancer types are indicated with orange asterisks and ICR disabled (HR>1, p-value<0.05) cancers are indicated with purple asterisks.

FIG. 5B depicts overall survival Kaplan-Meier curves of selected cancers, dichotomized by the enrichment score of the 3 ir-lncRNAs signature. Dichotomization cutoff of ‘high’ (red) and low′ (cyan) subgroups was based on optimal survival cutoff as determined by surv_cutpoint function. Cancer types in which the 3 ir-lncRNA signature holds significant prognostic value according to the logrank test were selected for visualization. Censor points are indicated by vertical lines. BRCA, breast invasive carcinoma; HNSC, head and neck squamous cell carcinoma; SKCM, skin cutaneous melanoma; UCEC, uterine corpus endometrial carcinoma; LIHC, liver hepatocellular carcinoma; CESC, cervical squamous cell carcinoma and endocervical adenocarcinoma; READ, rectum adenocarcinoma; GBM, glioblastoma multiforme; KIRP, kidney renal papillary cell carcinoma; KI RC, kidney renal clear cell carcinoma; LGG, brain lower grade glioma, RAQA, retrospective Arab Qatar cohort.

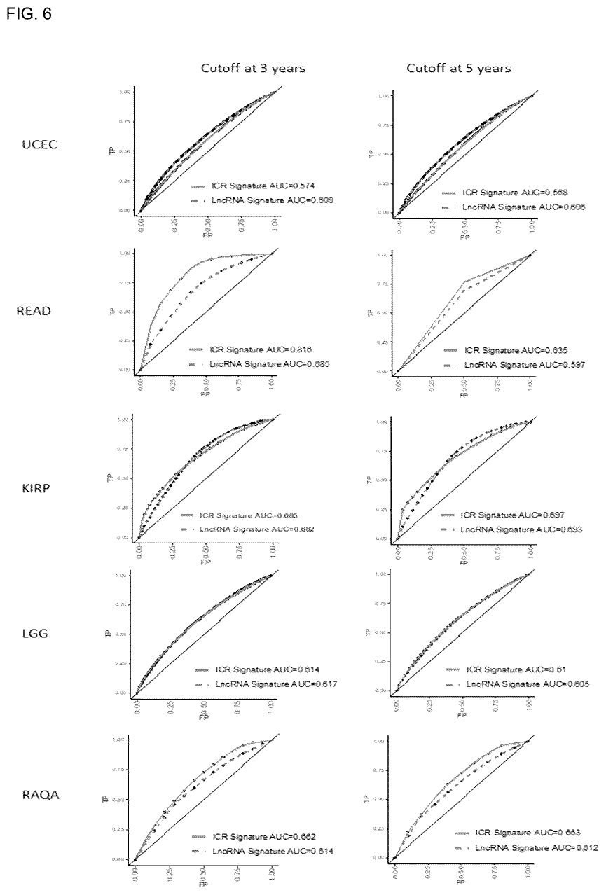

FIG. 6 depicts Receiver Operating Characteristic (ROC) analysis of 3 ir-lncRNA and ICR signatures in selected solid cancers. Time-dependent ROC curves of 3 ir-lncRNA (cyan) and ICR (red) signature with AUCs at three and five years. Cancer types for which 3 it-lcRNA signature showed the highest AUCs were selected for comparison with ICR signature AUC values. AUC, area under the curve; FP, false positive rate; KIRP, kidney renal papillary cell carcinoma; LGG, low-grade glioma, RAQA, retrospective Arab cohort Qatar; READ, rectum adenocarcinoma; ROC, receiver operating characteristic; TP, true positive rate; UCEC, uterine corpus endometrial carcinoma.

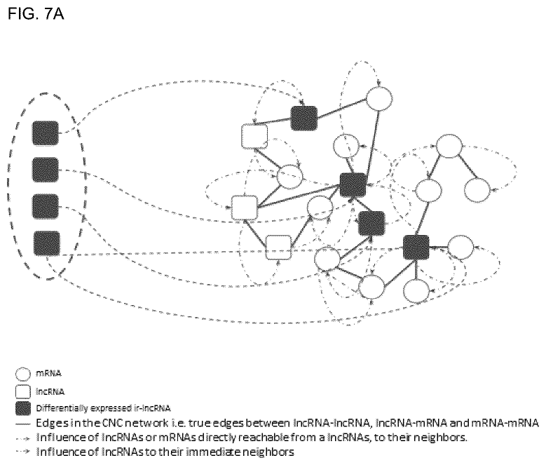

FIG. 7 depicts the mapping of differentially expressed ir-lncRNAs to protein coding genes.

FIG. 7A depicts a diagram representation of the random walk with restart global propagation network algorithm.

FIG. 7B depicts the walkscore distribution of protein-coding genes in TCGA-BRCA, with cutoff set at walkscore≥0.01 to generate a ranked list of protein-coding genes in proximity of differentially expressed ir-lncRNAs.

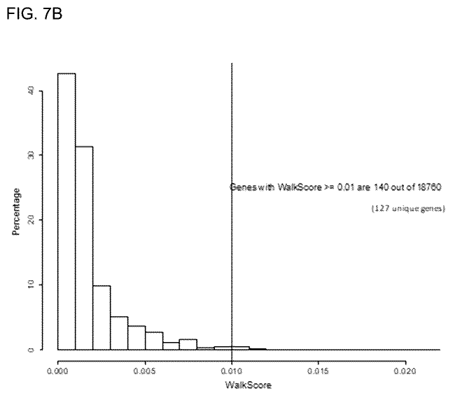

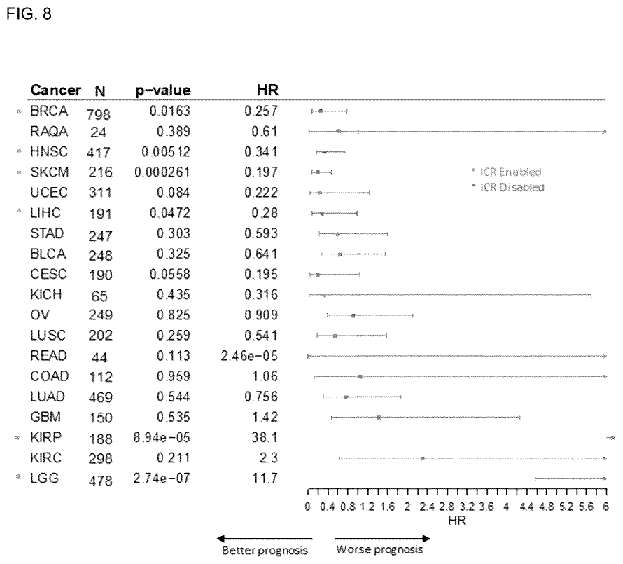

FIG. 8 depicts the prognostic value of the ICR classifier across solid cancers. Forest plot showing HRs (overall survival) of the continuous ICR score, p-values and number of patients for each TCGA cancer cohort and RAQA breast cancer cohort. Significant positive HRs are visualized in blue and significant negative HRs are visualized in red. ICR enabled (HR<1, p-value<0.05) cancer types are indicated with orange asterisks and ICR disabled (HR>1, p-value<0.05) cancers are indicated with purple asterisks.

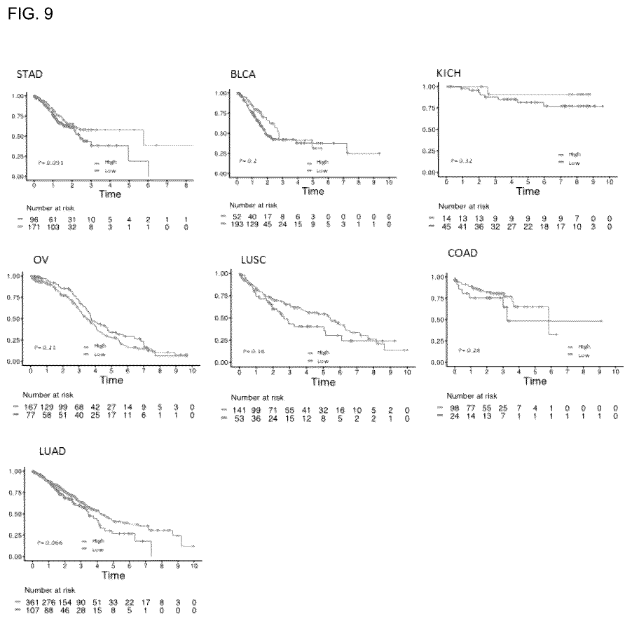

FIG. 9 depicts survival curves of the 3 ir-lncRNA signature. Overall survival Kaplan-Meier curves of ICR neutral cancers in which the 3 ir-lncRNAs signature did not show any significant prognostic value. Dichotomization cutoff of ‘high’ (red) and ‘low’ (cyan) subgroups was based on optimal survival cutoff as determined by surv_cutpoint function. Censor points are indicated by vertical lines. P-values were determined by logrank test.

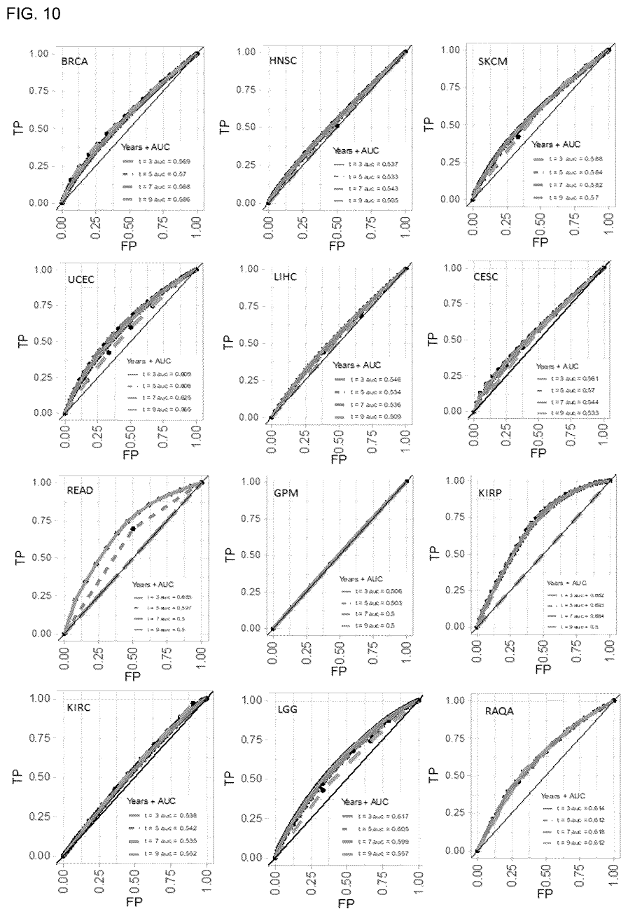

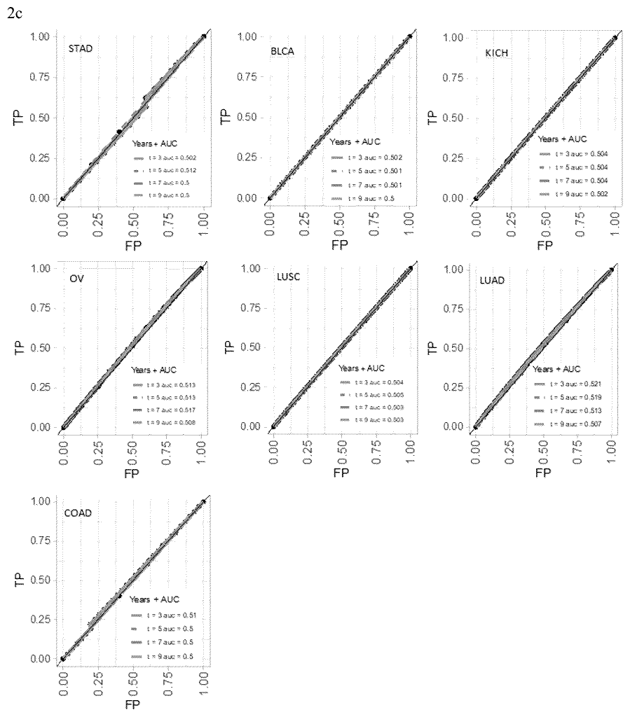

FIG. 10 depicts the ROC analysis of the 3 ir-lncRNA signature across multiple solid cancers. Time-dependent ROC curves of 3 ir-lncRNA signature with AUCs at different cutoffs; three, five, seven and nine years. ROC, receiver operating characteristic; AUC, area under the curve.

Disclosed embodiments can further comprise treatment of cancer, for example in patients whose immune related lncRNA signatures suggest the cancer is likely to progress.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### EXAMPLES

### Example 1. Workflow of Analysis

Few immune-related lncRNA signatures have been reported in cancer; however, their biological and clinical relevance and impact on downstream signaling pathways remain largely unexplored. To address this gap, we developed an analysis pipeline that involves the mapping of immune-related lncRNAs to coding-non-coding gene networks, followed by downstream analysis.

The analysis pipeline was first applied to the TOGA breast cancer dataset whereby key findings were validated in other TOGA cancer datasets. We opted to use the TOGA breast cancer dataset as a discovery cohort given its large sample size, detailed clinical annotation, and robust prognostic significance of the ICR gene signature. First, we applied a 2-step process to the TOGA breast cancer dataset by identifying differentially expressed lncRNAs in immune favorable versus unfavorable breast tumors, followed by determining their proximal coding genes and their likely downstream biological effects through pathways and correlation analyses (FIG. 1). Finally, the prognostic value of lncRNA signatures was explored across multiple TOGA cancer datasets in addition to a smaller breast cancer cohort from Qatar.

### Example 2. Identification of Differentially Expressed Ir-lncRNAs by ICR Tumor Immune Phenotype

TOGA breast tumor samples were classified into 3 subgroups based on the 20-gene ICR signature, and differentially expressed lncRNAs between ICR low (immune unfavorable) and ICR high (immune favorable) tumors were identified and labeled as immune related lncRNAs (ir-lncRNAs).

Out of a total of 12,727 lncRNAs, we identified 2988 to be differentially expressed (FDR p<0.05) of which 1284 were up- and 1704 were down-regulated in ICR high tumors (FIG. 2A). The top 5 ir-lncRNAs with the highest significant upregulation were HCP5, CTA-384D8, AC096579.7, LI NC01436 and WFDC21P and those with the 5 strongest significant downregulation included RP11-20F24.2, LINC00993, RP11-379F12.4, RP11-379F12.3 and RP11-53019.3 (Suppl Table 1).

### Example 3. Mapping of Ir-lncRNA to Proxy Coding Gene Networks

Next, we applied the RWR global propagation network algorithm to map the 2988 ir-lncRNAs to the CNC network and computed propagation scores to identify protein-coding genes within the network that are most likely influenced by the ir-lncRNAs. Based on the propagation scores, a ranked list of 127 unique protein-coding genes with walkscores 0.01 was compiled (Supp) Table 2).

We then performed limma analysis of these 127 protein-coding genes and could confirm that 37 and 40 were significantly up- and downregulated (FDR p<0.05) respectively (FIG. 2B, Suppl Table 3).

### Example 4. Biological Annotation of Protein-Coding Gene Networks Indicates Involvement in Immune and Metabolism Pathways

To gain insight into the putative downstream biological roles of the differentially expressed ir-lncRNAs, we explored enriched pathways, diseases and functions associated with the 127 protein-coding genes. Pathway enrichment analysis revealed that pathways involved in ‘Electron Transport Chain (OXPHOS system in mitochondria)’, ‘Respiratory electron transport’, ‘Oxidative phosphorylation’, ‘Complex I biogenesis’ and ‘Formation of ATP by chemiosmotic coupling’ were the most significantly enriched (p<0.05).

The first three pathways were mainly influenced by the differential expression of MT-ND1, MT-ND2, MT-CYB, NDUFB4, COX411, MT-ATP8 and MT-ATP6 genes (FIG. 2C, Suppl Table 4). Disease and function analysis identified several immunology related diseases and processes to be highly enriched in association with the 127 ir-lncRNA proxy protein-coding genes (p<0.05), such as ‘Immunological disease’, ‘Infectious diseases’, ‘Inflammatory disease’, ‘Inflammatory response’, ‘Immune cell trafficking’, ‘Humoral immune response’ and ‘Antimicrobial response’ in addition to ‘Cancer’ (FIG. 2D, Suppl Table 5).

### Example 5. Ir-lncRNAs are Associated with the Expression of Multiple Immune Checkpoints

In addition to its prognostic value, the ICR classifier serves as a predictor of response to immune checkpoint blockade. Furthermore, several lncRNAs have been found to be involved in the regulation of the immune checkpoint expression. Hence, we further explored the correlation of the differentially expressed ir-lncRNAs with immune checkpoints in the TCGA-BRCA cohort using Spearman correlation analysis (FIG. 3A, Suppl Table 6).

Overall, similar correlation patterns were observed between individual ir-lncRNAs and immune checkpoints. The strongest correlating immune checkpoints included CD40, IDO1, ICOS, CTLA4 and LAG3, whereas ADORA2A, VTCN1, CEACAM1 and TNFRSF14 showed the weakest correlations. Notably, CD276 (B7-H3) was the only immune checkpoint that was negatively correlated with ir-lncRNA expression.

### Example 6. Ir-lncRNA Signatures with Prognostic Value in Breast Cancer

Next, we investigated the prognostic value of two ir-lncRNA signatures in breast cancer, the first consisting of the top 20 differentially expressed ir-lncRNAs by ICR (20-ICRlncRNA) and the second of the top 20 differentially expressed ir-lncRNAs associated with immune checkpoints (20-ICPlncRNA). Both ir-lncRNA signatures conferred a significant survival benefit (20-ICRlncRNA [HR=0.2, p<0.01]; 20-ICPlncRNA [HR=0.304, p=0.0204]), with the 20-ICRlncRNA signature even outperforming the ICR score (HR=0.257, p=0.0163) (FIG. 3B).

Interestingly, the two ir-lncRNA signatures shared 3 common ir-lncRNAs (PCED1B-AS1, RP11-291B21.2 and AC092580.4, FIG. 3C) which together constitute a much smaller signature that retains prognostic significance (HR=0.359, p=0.0341) in a more practical format for clinical use.

### Example 7. Ir-lncRNA Signatures Demonstrate Prognostic Significance Across Multiple Tumor Types

Given the prognostic connotation of the ir-lncRNA signatures in breast tumors, we sought to assess its clinical value across different tumor types in comparison with the ICR classifier. For this purpose, we included TOGA datasets of 18 cancer types for which both gene and lncRNA expression data are available as well as one small breast cancer dataset from Qatar. Forest plot results (FIG. 4) show that both 20-lncRNA signatures are significantly associated with better overall survival in head and neck squamous cell carcinoma (HNSC, 20-ICRlncRNA HR=0.246 and 20-ICPlncRNA HR=0.312, p<0.01) and skin cutaneous melanoma (SKCM, 20-ICRlncRNA HR=0.226 and 20-ICPlncRNA HR=0.267, p<0.01) in addition to breast cancer (BRCA), whereas the opposite was true in kidney renal papillary cell carcinoma (KIRP, 20-ICRlncRNA [HR=11.2, p=0.0176], 20-ICPlncRNA [HR=36, p<0.01]) and low-grade glioma (LGG, 20-ICRlncRNA HR=37.1 and 20-ICPlncRNA HR=43.3, p<0.01). Furthermore, the 20-ICRlncRNA signature (FIG. 4A) was negatively correlated with overall survival in kidney renal clear cell carcinoma (KIRC, HR=21.7, p<0.01), while the 20-ICPlncRNA signature (FIG. 4B) was positively correlated with survival in uterine corpus endometrial carcinoma (UCEC, HR=0.188, p=0.0166), liver hepatocellular carcinoma (LIHC, HR=0.25, p=0.0439) and cervical squamous cell carcinoma and endocervical adenocarcinoma (CESC, HR=0.202, p=0.0328). In accordance with our previous work, we classified each tumor cohort with available lncRNA data as either ‘ICR enabled’ (HR<1 with a p-value<0.05), ‘ICR disabled’ (HR>1 with a p-value<0.05), or ‘ICR neutral’ (p-value>0.05) as based on the prognostic connotation of the ICR score (FIG. 8). Interestingly, all ICR-enabled tumors (BRCA, HNSC, SKCM, LIHC) were associated with a favorable prognostic ir-lncRNA signature and conversely, all ICR disabled tumors (KIRP, LGG) were characterized by an unfavorable prognostic ir-lncRNA signature.

### Example 8. Performance of 3 Ir-lncRNA Signature as a Prognostic Classifier in Cancer

Similarly, we assessed the prognostic value of the 3 ir-lncRNA signature across 18 tumor types (FIG. 5A) and found that it was associated with better prognosis in head and neck squamous cell carcinoma (HNSC, HR=0.34, p<0.01), skin cutaneous melanoma (SKCM, HR=0.302, p<0.01), uterine corpus endometrial carcinoma (UCEC, HR=0.158, p<0.01), and cervical squamous cell carcinoma and endocervical adenocarcinoma (CESC, HR=0.179, p=0.0487) in addition to breast cancer (BRCA, HR=0.359, p=0.0341). In contrast, the signature was strongly associated with a worse prognosis in kidney renal papillary cell carcinoma (KIRP, HR=95.4, p<0.01) and low-grade glioma (LGG, HR=13.7, p<0.01). Although no significant association was found in the Qatari breast cancer cohort, most likely to the small sample size, a clear trend for worse survival was observed (HR=0.181, ns). Comparing the prognostic performance of all three ir-lncRNA signatures with the ICR classifier shows that the 3 ir-lncRNA signature performs equally well as or even outperforms the 20 ir-lncRNA and ICR signatures with regards to its prognostic power in specific cancer types, applicability across a number of cancers and ease of use for clinical practice. Kaplan Meier survival curves with log-rank test (FIG. 5B, FIG. 8) corroborated the prognostic value of the 3 ir-lncRNA signature in several tumor types (ICR enabled [BRCA, HNSC, SKCM], ICR neutral [UCEC, CESC], ICR disabled [KIRP, LGG]) and showed additional significance in ICR enabled liver hepatocellular carcinoma (LIHC, p=0.05) and ICR neutral tumors such as rectum adenocarcinoma (READ, p<0.01), glioblastoma multiforme (GBM, p=0.013) and kidney renal clear cell carcinoma (KIRC, p=0.0026). Survival analysis of the RAQA breast cancer cohort showed a clear bifurcation of overall survival, albeit non-significant. Multivariate Cox regression analysis of the three individual ir-lncRNAs (Table 1) showed that out of the three lncRNAs, RP11-291B21.2 was most often associated with survival (STAD, GBM, KIRP, KIRC), followed by AC092580.4 (BRCA, STAD, LGG) and PCED1B-AS1 (LUAD, KIRP). Time-dependent ROC analysis was used to evaluate the sensitivity and specificity of the 3 ir-lncRNA signature in the various cancer cohorts and demonstrated overall similar areas under the curve for predicting 3-year, 5-year, 7-year or 9-year overall survival of cancer patients (FIG. 10). The 3 ir-lncRNA signature demonstrated the highest predictive power for overall survival in KIRP (3-year AUC=0.682, 5-year AUC=0.693) where the signature is strongly associated with worse survival (FIG. 6). The ROC curves for predicting 3-year overall survival showed the largest AUC in READ (AUC=0.685) and KIRP (AUC=0.682) followed by LGG (AUC=0.617), RAQA (AUC=0.614) and UCEC (AUC=0.609). At 5-year, the largest AUC was found in KIRP (AUC=0.693), RAQA (AUC=0.612), UCEC (AUC=0.606) and LGG (AUC=0.605). Compared with the ICR signature, the 3 lncRNA signature outperforms the ICR in UCEC (3-year AUC=0.609 vs 0.574, 5-year AUC=0.606 vs 0.568) and performs similarly in KIRP and LGG, whereas ICR has a larger AUC in READ (3-year AUC=0.816 vs 0.685, 5-year AUC=0.635 vs 0.597) and RAQA (3-year AUC=0.662 vs 0.614, 5-year AUC=0.663 vs 0.612).

TABLE 1
Multivariate analysis of the enrichment scores of the 3
ir-IncRNAs.

| | | | p | CI | CI | log | Cox |
|---|---|---|---|---|---|---|---|
| Cancer | Gene name | HR | value | lower | upper | rank p | p value |
| BRCA | AC092580.4 | [symbol] | [symbol] | [symbol] | [symbol] | [symbol] | [symbol] |
| | PCED1B-AS1 | | | | | | |
| | RP11-291B21.2 | | | | | | |
| HNSC | AC092580.4 | [symbol] | [symbol] | [symbol] | [symbol] | [symbol] | [symbol] |
| | PCED1B-AS1 | | | | | | |
| | RP11-291B21.2 | | | | | | |
| RAQA | AC092580.4 | [symbol] | [symbol] | [symbol] | [symbol] | [symbol] | [symbol] |
| | PCED1B-AS1 | | | | | | |
| | RP11-291B21.2 | | | | | | |
| SKCM | AC092580.4 | [symbol] | [symbol] | [symbol] | [symbol] | [symbol] | [symbol] |
| | PCED1B-AS1 | | | | | | |
| | RP11-291B21.2 | | | | | | |
| UCEC | AC092580.4 | — | — | — | — | [symbol] | [symbol] |
| | PCED1B-AS1 | [symbol] | [symbol] | [symbol] | [symbol] | | |
| | RP11-291B21.2 | — | — | — | — | | |
| LIHC | AC092580.4 | [symbol] | [symbol] | [symbol] | [symbol] | [symbol] | [symbol] |
| | PCED1B-AS1 | | | | | | |
| | RP11-291B21.2 | | | | | | |
| STAD | AC092580.4 | [symbol] | [symbol] | [symbol] | [symbol] | [symbol] | [symbol] |
| | PCED1B-AS1 | | | | | | |
| | RP11-291B21.2 | | | | | | |
| BLCA | AC092580.4 | [symbol] | [symbol] | [symbol] | [symbol] | [symbol] | [symbol] |
| | PCED1B-AS1 | | | | | | |
| | RP11-291B21.2 | | | | | | |
| CESC | AC092580.4 | [symbol] | [symbol] | [symbol] | [symbol] | [symbol] | [symbol] |
| | PCED1B-AS1 | | | | | | |
| | RP11-291B21.2 | | | | | | |
| KICH | AC092580.4 | [symbol] | [symbol] | [symbol] | [symbol] | [symbol] | [symbol] |
| | PCED1B-AS1 | | | | | | |
| | RP11-291B21.2 | | | | | | |
| OV | AC092580.4 | [symbol] | [symbol] | [symbol] | [symbol] | [symbol] | [symbol] |
| | PCED1B-AS1 | | | | | | |
| | RP11-291B21.2 | | | | | | |
| LUSC | AC092580.4 | [symbol] | [symbol] | [symbol] | [symbol] | [symbol] | [symbol] |
| | PCED1B-AS1 | | | | | | |
| | RP11-291B21.2 | | | | | | |
| READ | AC092580.4 | — | — | — | — | [symbol] | [symbol] |
| | PCED1B-AS1 | [symbol] | [symbol] | [symbol] | [symbol] | | |
| | RP11-291B21.2 | — | — | — | — | | |
| COAD | AC092580.4 | — | — | — | — | [symbol] | [symbol] |
| | PCED1B-AS1 | [symbol] | [symbol] | [symbol] | [symbol] | | |
| | RP11-291B21.2 | — | — | — | — | | |
| LUAD | AC092580.4 | [symbol] | [symbol] | [symbol] | [symbol] | [symbol] | [symbol] |
| | PCED1B-AS1 | | | | | | |
| | RP11-291B21.2 | | | | | | |
| GBM | AC092580.4 | [symbol] | [symbol] | [symbol] | [symbol] | [symbol] | [symbol] |
| | PCED1B-AS1 | | | | | | |
| | RP11-291B21.2 | | | | | | |
| KIRP | AC092580.4 | [symbol] | [symbol] | [symbol] | [symbol] | [symbol] | [symbol] |
| | PCED1B-AS1 | | | | | | |
| | RP11-291B21.2 | | | | | | |
| KIRC | AC092580.4 | [symbol] | [symbol] | [symbol] | [symbol] | [symbol] | [symbol] |
| | PCED1B-AS1 | | | | | | |
| | RP11-291B21.2 | | | | | | |
| LGG | AC092580.4 | [symbol] | [symbol] | [symbol] | [symbol] | [symbol] | [symbol] |
| | PCED1B-AS1 | | | | | | |
| | RP11-291B21.2 | | | | | | |

[symbol]

indicates data missing or illegible when filed

### Example 9. Cancer Treatment

An 18 year old patient is evaluated for cancer prognosis. After identification of at least one ir-lncRNA signature associated with cancer progression, the a treatment protocol including radiation therapy is prescribed.

### Example 10. Cancer Treatment

An 66 year old patient is evaluated for cancer prognosis. After identification of at least one ir-lncRNA signature associated with cancer progression, the a treatment protocol including immunotherapy is prescribed.

### Example 11. Cancer Treatment

An 54 year old patient is evaluated for cancer prognosis. After identification of at least one ir-lncRNA signature associated with cancer progression, the a treatment protocol including chemotherapy is prescribed.

### DISCLOSED EMBODIMENTS

Embodiment 1—A method for predicting clinical outcome in cancer, the method comprising of identification of at least one ir-lncRNA signature, wherein said at least one signature is associated with cancer progression.

Embodiment 2—The method of embodiment 1, wherein the ir-lncRNA signature comprises three lncRNAs.

Embodiment 3—The method of embodiment 1, wherein the ir-lncRNA signature comprises the top 20 differentially expressed ir-lncRNAs in ICR high versus ICR low breast tumors

Embodiment 4—The method of embodiment 1, wherein the ir-lncRNA signature comprises the top 20 ir-lncRNAs that are positively correlated with immune checkpoint expression.

Embodiment 5—The method of embodiment 1, wherein the cancer comprises breast invasive carcinoma, head and neck squamous cell carcinoma, skin cutaneous melanoma, uterine corpus endometrial carcinoma, liver hepatocellular carcinoma, cervical squamous cell carcinoma and endocervical adenocarcinoma, kidney renal papillary cell carcinoma, kidney renal clear cell carcinoma, or low grade glioma.

Embodiment 6—The method of embodiment 5, wherein the cancer comprises one or any combination of the above-mentioned cancers.

Embodiment 7—A method of correlating immune checkpoint expression with one ir-lncRNA signature or a combination of ir-lncRNA signatures, the method comprising of identification of at least one ir-lncRNA signature correlated with immune checkpoint expression.

Embodiment 8—The method of embodiment 7, wherein the ir-lncRNA signature comprises three lncRNAs.

Embodiment 9—The method of embodiment 7, wherein the ir-lncRNA signature comprises the top 20 differentially expressed ir-lncRNAs in ICR high versus ICR low breast tumors

Embodiment 10—The method of embodiment 7, wherein the ir-lncRNA signature comprises the top 20 ir-lncRNAs that are positively correlated with immune checkpoint expression.

Embodiment 11—The method of any preceding embodiment, wherein said ir-lncRNA signature comprises at least one of PCED1B-AS1, RP11-291B21.2 and AC092580.4.

Embodiment 12—The method of any preceding embodiment, further comprising development of a cancer treatment protocol for the patient.

Embodiment 13—The method of embodiment 12, further comprising administration of a cancer treatment comprising at least one of chemotherapy, radiation therapy, and immunotherapy.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for predicting clinical outcome in cancer, the method comprising of identification of at least one ir-lncRNA signature, wherein said at least one signature is associated with cancer progression.

2. The method of claim 1, wherein the ir-lncRNA signature comprises three lncRNAs.

3. The method of claim 1, wherein the ir-lncRNA signature comprises the top 20 differentially expressed ir-lncRNAs in ICR high versus ICR low breast tumors

4. The method of claim 1, wherein the ir-lncRNA signature comprises the top 20 ir-lncRNAs that are positively correlated with immune checkpoint expression.

5. The method of claim 1, wherein the cancer comprises breast invasive carcinoma, head and neck squamous cell carcinoma, skin cutaneous melanoma, uterine corpus endometrial carcinoma, liver hepatocellular carcinoma, cervical squamous cell carcinoma and endocervical adenocarcinoma, kidney renal papillary cell carcinoma, kidney renal clear cell carcinoma, or low grade glioma.

6. The method of claim 5, wherein the cancer comprises one or any combination of the above-mentioned cancers.

7. A method of correlating immune checkpoint expression with one ir-lncRNA signature or a combination of ir-lncRNA signatures, the method comprising of identification of at least one ir-lncRNA signature correlated with immune checkpoint expression.

8. The method of claim 7, wherein the ir-lncRNA signature comprises three lncRNAs.

9. The method of claim 7, wherein the ir-lncRNA signature comprises the top 20 differentially expressed ir-lncRNAs in ICR high versus ICR low breast tumors

10. The method of claim 7, wherein the ir-lncRNA signature comprises the top 20 ir-lncRNAs that are positively correlated with immune checkpoint expression.

11. The method claim 1, wherein said ir-lncRNA signature comprises at least one of PCED1B-AS1, RP11-291B21.2 and AC092580.4.

12. The method of claim 11, further comprising development of a cancer treatment protocol for the patient.

13. The method of claim 12, further comprising administration of a cancer treatment comprising at least one of chemotherapy, radiation therapy, and immunotherapy.
